(19) United States
(12) Patent Application Publication
(10) Pub. No.: US 2026/0016403 A1
(43) Pub. Date: Jan. 15, 2026

(54) SOLID-STATE IMAGING DEVICE AND SOLID-STATE IMAGING SYSTEM

(71) Applicant: Sony Semiconductor Solutions Corporation, Kanagawa (JP)

(72) Inventor: Yusuke Yokoyama, Kanagawa (JP)

(21) Appl. No.: 18/995,041

(22) PCT Filed: Jun. 20, 2023
(86) PCT No.: PCT/JP2023/022696

§ 371 Date: Jan. 15, 2025

(30) Foreign Application Priority Data
Aug. 9, 2022 (JP) 2022-127350

(51) Int. Cl. G01N 21/3554; G01N 21/359; G01N 21/88; G01N 31/22; G01N 33/02

(52) U.S. Cl. CPC G01N 21/3554; G01N 21/8851; G01N 31/222; G01N 33/02; G01N 21/359

(57) ABSTRACT

Solid-state imaging configured to acquire information regarding moisture content together with appearance data is disclosed. In one example, a solid-state imaging device includes an imaging element and a processing circuit. The imaging element includes, in a single pixel array, a visible-light receiving element that receives visible light and an infrared-light receiving element that receives infrared light in at least a short-wavelength infrared band. The processing circuit generates a first image illustrating an image of a dried food in a visible region and a second image illustrating an image of the dried food in an infrared region from data acquired by the imaging element, and inspects the dried food from the first image and the second image.

### TECHNICAL FIELD

The present disclosure relates to a solid-state imaging device and a solid-state imaging system.

### BACKGROUND ART

In an image sensor capable of imaging in a near-infrared band, it is possible to detect moisture that is difficult to distinguish with visible light by using a wavelength band indicating absorption of water. In a process of manufacturing a dried article such as a dry food by using this characteristic, moisture remaining in the article is detected using a line sensor capable of receiving light in a near-infrared band.

However, for example, in a quality inspection process of a dry food, it is necessary to perform detailed moisture management and to detect occurrence of an appearance abnormality due to drying or the like, and a skilled person detects the occurrence, and moreover, determines whether or not moisture remains. As a result, there are problems such as the inspection efficiency and dependency on individual skills in making the determination.

### CITATION LIST

### Patent Document

Patent Document 1: Japanese Patent Application Laid-Open No. 2016-017932

### SUMMARY OF THE INVENTION

### Problems to be Solved by the Invention

Therefore, the present disclosure provides a solid-state imaging device that acquires information regarding a moisture content together with appearance data.

### Solutions to Problems

According to an embodiment, a solid-state imaging device includes an imaging element and a processing circuit. The imaging element includes, in a single pixel array, a visible-light receiving element that receives visible light and an infrared-light receiving element that receives infrared light in at least a short-wavelength infrared band. The processing circuit generates a first image illustrating an image of a dried food in a visible region and a second image illustrating an image of the dried food in an infrared region from data acquired by the imaging element, and inspects the dried food from the first image and the second image that have been generated.

The processing circuit may detect a moisture content of the dried food from at least the second image.

The processing circuit may compare the moisture content with a predetermined threshold and issue a notification that the dried food having the moisture content higher than the predetermined threshold is subject to a re-drying process.

The processing circuit may calculate a drying time in the re-drying process on the basis of the moisture content.

The processing circuit may infer the moisture content of the dried food by using a learned model.

The processing circuit may detect a protein content of the dried food from at least the second image.

The processing circuit may infer the protein content of the dried food by using a learned model.

The processing circuit may detect a defect in appearance of the dried food from at least the first image.

The processing circuit may detect foreign matter attached to the dried food from the first image.

The processing circuit may detect deterioration of a color tone of the dried food from the first image.

The processing circuit may perform an appearance inspection of the dried food by using a learned model.

The processing circuit may inspect the dried food on the basis of the first image and the second image by using a learned model.

According to an embodiment, a solid-state imaging system includes an infrared light source and a solid-state imaging device. The infrared light source emits infrared light including at least a short-wavelength infrared band. The solid-state imaging device is the solid-state imaging device according to any one of the above. The imaging element causes the infrared-light receiving element to receive light emitted from the infrared light source and reflected by or transmitted through the dried food.

### BRIEF DESCRIPTION OF DRAWINGS

FIG. 1 is a diagram schematically illustrating an example of a solid-state imaging device according to an embodiment.

FIG. 2 is a diagram schematically illustrating an example of a pixel array of the solid-state imaging device according to an embodiment.

FIG. 3 is a flowchart illustrating an example of processing by the solid-state imaging device according to an embodiment.

FIG. 4 is a diagram illustrating an example of an area to be imaged according to an embodiment.

FIG. 5 is a diagram illustrating examples of a first image and a second image according to an embodiment.

FIG. 6 is a diagram illustrating an example of an image according to an embodiment.

FIG. 7 is a diagram schematically illustrating an example of a solid-state imaging system according to an embodiment.

### MODE FOR CARRYING OUT THE INVENTION

Hereinafter, embodiments of the present disclosure will be described with reference to the drawings. The drawings are used for explanation, and the shape and size of each component in actual devices, the ratios of size to other components, and the like are not necessarily as illustrated in the drawings. Furthermore, since the drawings are illustrated in a simplified manner, it should be understood that components necessary for implementation other than those illustrated in the figure are provided as appropriate.

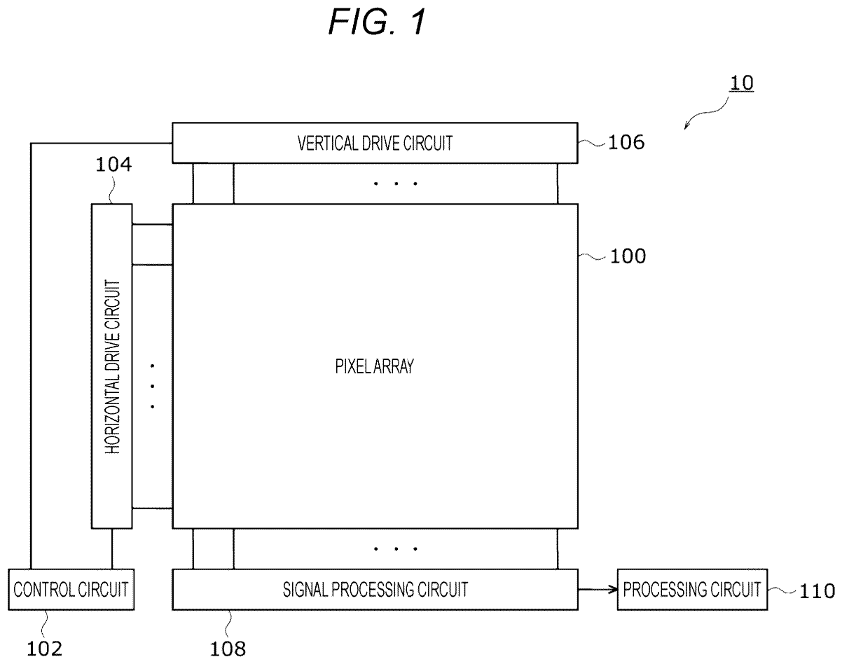

FIG. 1 is a diagram schematically illustrating an example of a solid-state imaging device according to an embodiment. A solid-state imaging device 10 includes a pixel array 100, a control circuit 102, a horizontal drive circuit 104, a vertical drive circuit 106, a signal processing circuit 108, and a processing circuit 110. The solid-state imaging device 10 is, for example, a device that acquires a first image and a second image of an object in the same range, the first image being a visible-light image and the second image being an infrared-light image, and appropriately processes these images.

The pixel array 100 is a region in which light receiving pixels having light receiving elements are arranged in a two-dimensional array. Each of the light receiving pixels is connected to a pixel circuit. The light receiving element outputs a signal corresponding to the intensity of incident light by photoelectric conversion, and the pixel circuit appropriately converts the signal output from the light receiving element into a signal for each pixel and outputs the signal. By acquiring reflected light or the like of an object via the pixel array 100, the solid-state imaging device 10 can operate as an area sensor.

The control circuit 102 is a circuit that controls signal acquisition of the solid-state imaging device 10. The control circuit 102 performs control to output a signal obtained by photoelectric conversion in the pixel array 100 to the signal processing circuit 108 by the horizontal drive circuit 104 and the vertical drive circuit 106. Note that the control circuit 102 may also be connected to the signal processing circuit 108.

Under the control of the control circuit 102, the horizontal drive circuit 104 selects a line of the pixel array 100 and performs driving in which a signal is output from a light receiving pixel belonging to the line.

Under the control of the control circuit 102, the vertical drive circuit 106 drives a light receiving pixel belonging to a column at an appropriate timing of a light receiving pixel belonging to the line selected by the horizontal drive circuit 104, and performs control to output a signal.

The signal processing circuit 108 appropriately processes analog signals output from the respective light receiving pixels and outputs the signals. The signal processing circuit 108 may include, for example, a column analog-to-digital converter (ADC) that performs analog-to-digital (AD) conversion for each column, and may convert an analog signal output from the light receiving pixel into a digital signal and output the digital signal. The signal processing circuit 108 converts the digital signal into, for example, an image signal by another appropriate circuit and outputs the image signal.

The processing circuit 110 performs appropriate processing on the image data output from the signal processing circuit 108. For example, the processing circuit 110 may perform image processing on the image data, and executes information processing such as classification by a rule-based method or classification using a learned model.

Note that, although not illustrated, the solid-state imaging device 10 may include a storage circuit. The storage circuit can appropriately store, for example, image data in the signal processing circuit 108 or the processing circuit 110. Furthermore, in a case where at least part of processing by the signal processing circuit 108 or the processing circuit 110 is specifically implemented by information processing by software using hardware resources, the storage circuit may store a program, an execution file, and the like necessary for the information processing by the software. In this case, at least a part of the processing by the signal processing circuit 108 or the processing circuit 110 may be realized by referring to a program or the like by each circuit, the program or the like being stored in the storage circuit.

In FIG. 1, the processing circuit 110 is provided inside the solid-state imaging device 10, but the present invention is not limited thereto. For example, the processing circuit 110 may be provided outside the solid-state imaging device 10. For example, in the solid-state imaging device 10, the pixel array 100, the control circuit 102, the horizontal drive circuit 104, the vertical drive circuit 106, the signal processing circuit 108, and the processing circuit 110 may be formed on the same semiconductor chip, or at least some of the components may be formed on another semiconductor chip.

In a case where these components are formed on the same semiconductor chip, these components may be formed in stacked semiconductor layers, and the layers may be electrically connected by an any method such as a microbump or a via hole. In a case where the semiconductor chips are formed by being stacked, the semiconductor chips may be stacked by any method such as chip-on-chip (CoC), chip-on-wafer (CoW), or wafer-on-wafer (Wow) after individual layers are formed, or necessary components may be sequentially formed on one substrate.

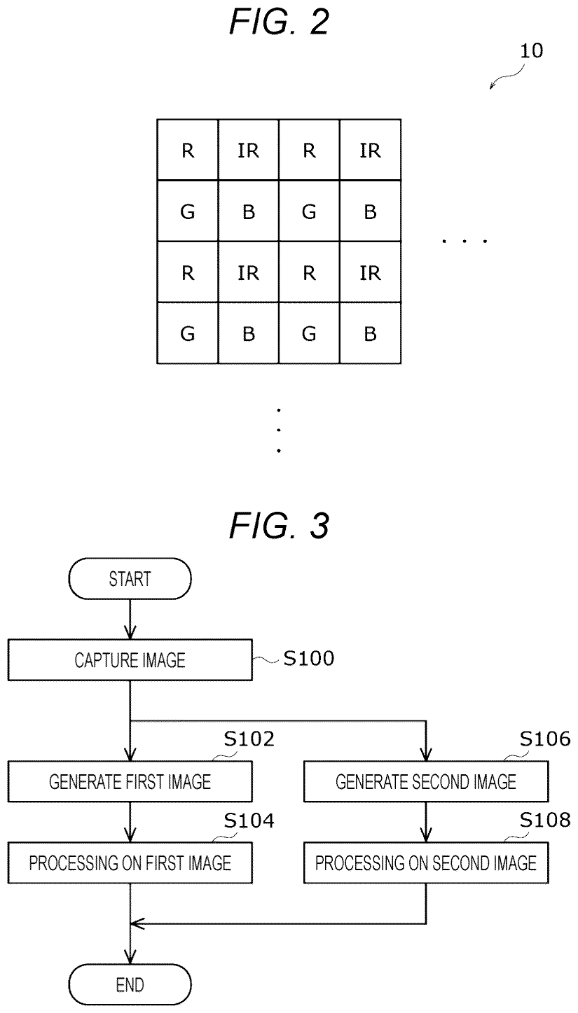

FIG. 2 is a diagram illustrating a non-limiting example of arrangement of pixels in the pixel array 100 according to an embodiment. R means that an element that receives red light is provided, G means that an element that receives green light is provided, B means that an element that receives blue light is provided, and IR means that an element that receives infrared light is provided. FIG. 2 may be arrangement of pixels or arrangement of divided photoelectric conversion regions (divided pixels) in a pixel.

As an example, in the pixel array 100, as illustrated in FIG. 2, some light receiving elements in the Bayer array may be light receiving elements capable of receiving infrared light. The original arrangement may not be the Bayer arrangement, and a light receiving element that receives light in other than the three primary colors of RGB may be provided.

Here, in the light receiving element region indicated by IR, a mode of being able to receive light in at least a short-wavelength infrared (SWIR) band is possible. Furthermore, as another example, a light receiving element capable of receiving light in a near-infrared (NIR) band may be provided in addition to an element that receives SWIR light.

Each of the light receiving elements that receive light in the bands of the respective colors may be in a mode of including, for example, a color filter on the incident surface side of the light receiving element, or may be in a mode of including an organic photoelectric conversion element. That is, designation of the band of light to be received in each light receiving element may be implemented by any method.

Furthermore, it is assumed that the pixel receives light in the visible-light band and the infrared-light band as described above; however, arrangement of a pixel that acquires other types of information is not excluded. As some non-limiting examples, the pixel array 100 may include, at least in part, a pixel capable of acquiring an any polarization state, a pixel capable of acquiring an image plane phase difference, a pixel capable of acquiring time-of-flight (ToF) information, a pixel capable of detecting event information, a pixel using a plasmon filter, or the like.

In the pixel array 100, for example, by adopting arrangement of the light receiving elements illustrated in FIG. 2, the solid-state imaging device 10 can generate images in the visible-light band and the infrared-light band for the same target information. That is, the solid-state imaging device 10 can acquire a first image that is an image in the visible-light band and a second image that is an image in the infrared-light band for the same target.

That is, the solid-state imaging device 10 operates as an area sensor that images the same region in the visible-light band and the infrared-light band. As described above, since the coordinates of the first image and the second image are basically not shifted, the solid-state imaging device 10 can acquire visible information and infrared information of the same target at the same coordinates.

Hereinafter, as a non-limiting example, acquiring an image of a dry food (dried food) and performing information processing will be described. However, the solid-state imaging device 10 according to the present disclosure is not applied only to a dry food, and can execute similar processing on any target. Furthermore, as an example, an image in the SWIR band is acquired as the second image, but this band can be changed according to the purpose.

The data on the first image and the second image acquired by the signal processing circuit 108 is transmitted to the processing circuit 110, and the processing circuit 110 can realize any processing on these images.

FIG. 3 is a flowchart illustrating processing by the solid-state imaging device 10 according to an embodiment.

The solid-state imaging device 10 converts light in the visible-light band and light in the infrared-light band in a region where the pixel array 100 can receive light into signals and outputs the signals (S100). For example, the solid-state imaging device 10 images a region where a food which has undergone drying processing exists in a dry food, receives light reflected by or transmitted through the dried food by the light receiving elements in pixel array 100, and outputs light for each pixel.

The signal processing circuit 108 converts analog signals output from the pixel circuits into digital signals, and generates a first image by performing appropriate processing on light reception signals in the visible-light band (S102). For example, on the basis of the signals acquired from the light receiving elements corresponding to the R, G, and B wavelength bands in FIG. 2, the signal processing circuit 108 appropriately mixes the outputs, and acquires the first image, which is an image in the visible-light band.

The processing circuit 110 executes processing on the first image generated by the signal processing circuit 108 (S104). This processing is, for example, processing of inspecting the appearance of a dried food. For example, the processing circuit 110 inspects whether or not a defect is generated in the appearance of the dried food illustrated in the first image on the basis of the acquired color information.

For example, by referring to the first image, the processing circuit 110 can detect foreign matter attached to the dried food and/or detect degradation or deterioration of the color tone of the dried food. For example, the processing circuit 110 can notify the user or the like to perform the inspection again for the dried food in which foreign matter or degradation or deterioration of the color tone is detected.

Furthermore, a mode is possible in which on the basis of output of the processing circuit 110, a defective product removal module or the like outside the solid-state imaging device 10 automatically removes a dried food in which foreign matter, degradation, or deterioration is detected in a lane of a factory, or executes reinspection by a user, for example.

In parallel with the above processing, the solid-state imaging device 10 may execute acquisition processing of a second image, which is an image in the SWIR band and processing on the second image. The processing may not be performed in parallel. For example, the processing of the second image may be performed after the processing of the first image is completed, or the processing of the first image may be performed after the processing of the second image is completed.

The signal processing circuit 108 converts analog signals output from the pixel circuits into digital signals, and generates a second image by performing appropriate processing on light reception signals in the SWIR band (S106). For example, on the basis of the signals acquired from the light receiving elements corresponding to the IR wavelength band in FIG. 2, the signal processing circuit 108 acquires the second image, which is an image in the SWIR band from the outputs.

The processing circuit 110 executes processing on the second image generated by the signal processing circuit 108 (S108). This processing is, for example, processing of inspecting the moisture content of the dried food. The light in the SWIR band has a characteristic of being absorbed by moisture (water). Therefore, for example, the processing circuit 110 can detect the moisture content of the dried food by referring to the second image.

For example, the processing circuit 110 may compare the moisture content of the dried food with a predetermined threshold by referring to the second image, and in a case where the moisture content is higher than the predetermined threshold, may notify the user to re-dry the dried food, or may perform processing such that the re-drying process is automatically performed on the dried food that needs to be re-dried from, for example, a factory lane or the like in an external re-drying process performing module or the like.

The predetermined threshold need not be one, and a plurality of thresholds may be provided. Furthermore, the moisture content may be acquired as a continuous value.

The processing circuit 110 may determine the presence or absence of the re-drying process from the moisture content of the dried food, and moreover, may calculate and output the drying time in the re-drying process of the dried food. As described above as an example, in a case where the moisture content is determined by a threshold, the processing circuit 110 may set the drying time in the determined range, or in a case where the moisture content is acquired as a continuous value, the processing circuit 110 may calculate the drying time for the continuous value.

As another example, the processing circuit 110 may detect a component absorbed in light in the SWIR band from the acquired second image. For example, the processing circuit 110 can acquire the protein content of the dried food from the second image. The processing circuit 110 can also infer the moisture content of the dried food from the protein content.

As described above, according to the solid-state imaging device 10 of the present embodiment, the first image in the visible-light band and the second image in the SWIR band can be acquired at the same timing (alternatively, the timing may be different for a stationary object) for the target existing in the same region. Therefore, it is not necessary to execute processing related to the solid-state imaging device 10 such as optical axis alignment, and it is possible to perform appropriate processing using the respective pieces of information in the visible-light band and the SWIR band by using the same coordinates.

As in the non-limiting example described above, according to the solid-state imaging device 10, it is possible to detect a flaw or the like suitable for visible light in a visible light image and acquire information such as a moisture content of a dry object in an SWIR image. Using the characteristic of SWIR, as another example, the solid-state imaging device 10 can nondestructively inspect the inside of an object including resin that transmits light in the SWIR band at the same timing as that for the appearance.

Furthermore, the band of infrared light to be received is not limited to SWIR, and a light receiving element that receives light in the NIR band may be further provided. Furthermore, light receiving elements that receive light of a plurality of wavelength regions in the SWIR band may be provided. In such a case, it is also possible to detect flaws and the like regarding different characteristics from images having the different characteristics in the band of infrared light. For example, in the NIR band, it is possible to detect foreign matter that cannot be found by an appearance inspection in a region containing moisture and it is also possible to detect the moisture content in the SWIR band.

In the above embodiment, the overall processing flow has been described. Hereinafter, the processes of inspection by the processing circuit 110 will be more specifically described.

The processing circuit 110 can also acquire the moisture content for each type of dry food from the second image. For example, the processing circuit 110 may receive in advance data regarding the type of food to be imaged by the solid-state imaging device 10, extract data indicating a relationship between the moisture content and color density from a data sheet or the like on the basis of the type of food, and detect the moisture content from the second image on the basis of the data.

The processing circuit 110 can execute determination processing on the first image by using a learned model. This learned model is, for example, a model trained by machine learning by using, as training data, images (visible-light images) of a target food to which labels of a non-defective product and a defective product visually determined by a human are attached. The labels are not limited to non-defective/defective, and a plurality of labels such as no defect, foreign matter adhesion, and poor color tone may be attached.

This model may be trained by any machine learning method. This model may determine (classify) whether or not there is a defect in each image, or may determine whether or not there is a defect in a partial region (target food) in the image.

That is, according to the solid-state imaging device 10 of the present disclosure, it is also possible to capture images of a plurality of targets and implement various inspections on the plurality of captured images at the same timing. In this case, with respect to the target for which it is determined that a defect has occurred, it is possible to acquire information on which target the defect has occurred in each of the first image and the second image by referring to the coordinates or the address.

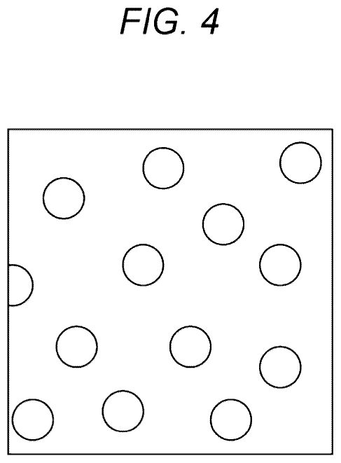

FIG. 4 is a diagram illustrating an example of an area to be imaged. A circle in the figure indicates a target object. The solid-state imaging device 10 acquires a first image and a second image with respect to an image including a plurality of such targets.

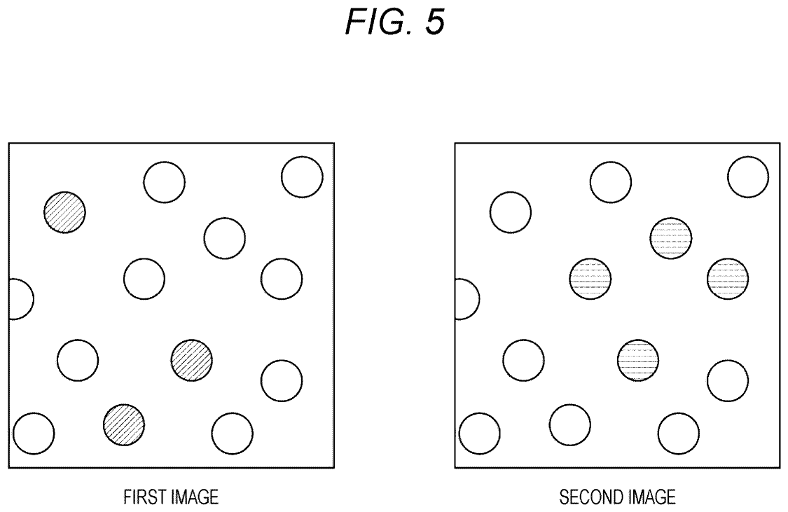

FIG. 5 is a diagram illustrating examples of the first image and the second image with respect to FIG. 4 described above. The left figure illustrates an example of the first image, and the right figure illustrates an example of the second image.

For example, it is assumed that the processing circuit 110 detects that a target indicated by hatching in the first image has a defective appearance and detects moisture in a target indicated by horizontal lines in the second image. As described above, the solid-state imaging device 10 according to the present disclosure can detect a defective target for each target in each of the first image and the second image.

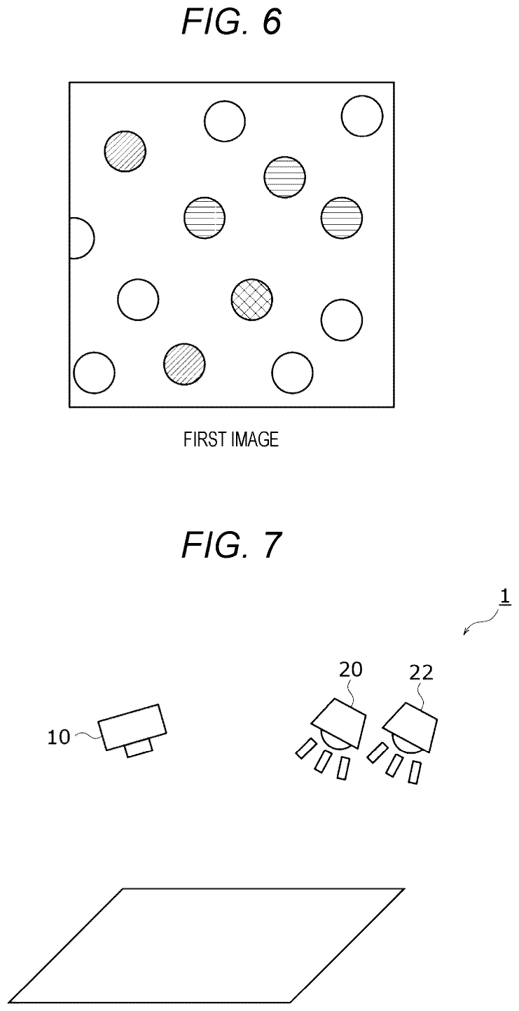

FIG. 6 is a diagram illustrating an image in which the targets determined to be defective in the second image are reflected in the first image. The processing circuit 110 can also aggregate (combine) the defective targets determined in the respective images as illustrated in FIG. 5 into one image. For example, the processing circuit 110 can indicate a target having a defective appearance and a target containing moisture on the same image by marking the target determined to be defective in the second image on the first image.

In FIG. 6, as an example, the processing circuit 110 marks the image determined to be defective in both the first image and the second image differently from the targets determined to be defective in the respective images. Since information is acquired using the pixels included in the same pixel array 100 by using the same optical system, according to the solid-state imaging device 10, such image synthesis can also be easily achieved. Note that, for example, in the case of display output on a display device, the marking can be indicated by changing the color of the target, blinking the target, surrounding the target with a circle, or the like, or can be indicated as “defective appearance”, “moisture content: large”, or the like as character information for the target.

Furthermore, the model may be in any mode as long as the model can appropriately perform determination, and as a non-limiting example, the model may be multi-layer perceptron (MLP) or in a mode including a convolutional layer in at least a part thereof.

Furthermore, regarding the second image, it is often possible to make a judgement based on color density. Therefore, the processing circuit 110 may perform rule-based determination, or similarly to the above, may use a learned model separately learned, also for the second image. That is, the processing circuit 110 may determine or infer the moisture content (including the presence or absence of the moisture content) and/or the protein content (including the presence or absence of the protein content) with respect to the second image by using the learned model.

In the above description, either the first image or the second image is input to the learned model, but the learned model is not limited thereto. The learned model may be, for example, in a mode in which data of the first image and the second image is input from an input layer, and inspection (inspection of both appearance inspection and inspection of moisture content or the like) is performed on a target such as a dried food from information on both the input images.

In this case, a mode is also possible in which the signal processing circuit 108 can output digital data for each color (including the IR band) output from the respective pixel circuits, and the processing circuit 110 can input the data for each color output from the signal processing circuit 108 to the learned model.

The solid-state imaging device 10 can appropriately acquire the first image by capturing an image in a state in which a human can sense a target, for example, in a state in which a target is irradiated with sunlight or light of a fluorescent lamp or the like. On the other hand, it is unclear whether or not the target is irradiated with light in a desired infrared-light band. Therefore, the solid-state imaging device 10 may form a solid-state imaging system together with a light source that emits at least light in an infrared-light band.

FIG. 7 is a diagram schematically illustrating an example of a solid-state imaging system according to an embodiment. A solid-state imaging system 1 includes at least the solid-state imaging device 10 and an infrared light source 20. The solid-state imaging system 1 may further include a visible light source 22.

The infrared light source 20 is, for example, a light source that irradiates a target with infrared light. The infrared light source 20 is, for example, a light source that irradiates a target with light including a wavelength in an SWIR band capable of generating an image in the solid-state imaging device 10. In a case where the solid-state imaging device 10 can acquire information on a wavelength in an NIR band together with a wavelength in an SWIR band, the infrared light source 20 may be a light source including a wavelength in the NIR band, or a light source corresponding to a wavelength in the NIR band may be separately provided. Furthermore, in a case where the solid-state imaging device 10 can acquire a plurality of SWIR bands separately, the infrared light source 20 may be a light source including light of wavelengths in all of these bands.

The solid-state imaging system 1 can generate an image by receiving light in a desirable wavelength band in the solid-state imaging device 10 by using the infrared light source 20 to irradiate food or the like to be inspected with infrared light.

Note that, as described above, the solid-state imaging system 1 may further include the visible light source 22. The visible light source 22 is a light source including a wavelength in a band in which an appropriate first image can be acquired in the solid-state imaging device 10.

According to the solid-state imaging system 1 of FIG. 7, it is possible to generate an image based on light in a desirable wavelength band in the solid-state imaging device 10. As a result, according to the solid-state imaging system 1, it is possible to accurately implement the inspection in the solid-state imaging device 10 described above.

The embodiments described above may have the following modes.

(1)

A solid-state imaging device including:
an imaging element including, in a single pixel array, a visible-light receiving element that receives visible light and an infrared-light receiving element that receives infrared light in at least a short-wavelength infrared band; and
a processing circuit that generates a first image illustrating an image of a dried food in a visible region and a second image illustrating an image of the dried food in an infrared region from data acquired by the imaging element, and inspects the dried food from the first image and the second image that have been generated.
   (2)

The solid-state imaging device according to (1), in which
the processing circuit detects a moisture content of the dried food from at least the second image.
   (3)

The solid-state imaging device according to (2), in which
the processing circuit compares the moisture content with a predetermined threshold and issues a notification that the dried food having the moisture content higher than the predetermined threshold is subject to a re-drying process.
   (4)

The solid-state imaging device according to (3), in which
the processing circuit calculates a drying time in the re-drying process on the basis of the moisture content.
   (5)

The solid-state imaging device according to any one of (2) to (4), in which
the processing circuit infers the moisture content of the dried food by using a learned model.
   (6)

The solid-state imaging device according to any one of (1) to (5), in which
the processing circuit detects a protein content of the dried food from at least the second image.
   (7)

The solid-state imaging device according to (6), in which
the processing circuit infers the protein content of the dried food by using a learned model.
   (8)

The solid-state imaging device according to any one of (1) to (7), in which
the processing circuit detects a defect in appearance of the dried food from at least the first image.
   (9)

The solid-state imaging device according to (8), in which
the processing circuit detects foreign matter attached to the dried food from the first image.
   (10)

The solid-state imaging device according to (8) or (9), in which
the processing circuit detects deterioration of a color tone of the dried food from the first image.
   (11)

The solid-state imaging device according to any one of (8) to (10), in which
the processing circuit performs an appearance inspection of the dried food by using a learned model.
   (12)

The solid-state imaging device according to any one of (1) to (11), in which
the processing circuit inspects the dried food on the basis of the first image and the second image by using a learned model.
   (13)

A solid-state imaging system including:
an infrared light source that emits infrared light including at least a short-wavelength infrared band; and
the solid-state imaging device according to any one of (1) to (12),
in which the imaging element causes the infrared-light receiving element to receive light emitted from the infrared light source and reflected by or transmitted through the dried food.

Aspects of the present disclosure are not limited to the above-described embodiments, and include various conceivable modifications. The effects of the present disclosure are not limited to the above-described contents. The components in each of the embodiments may be appropriately combined and applied. That is, various additions, modifications, and partial deletions can be made without departing from the conceptual idea and gist of the present disclosure derived from the contents defined in the claims and equivalents and the like thereof.

### REFERENCE SIGNS LIST

1 Solid-state imaging system
10 Solid-state imaging device
100 Pixel array
102 Control circuit
104 Horizontal drive circuit
106 Vertical drive circuit
108 Signal processing circuit
110 Processing circuit
20 Infrared light source
22 Visible light source

1. A solid-state imaging device comprising:
an imaging element including, in a single pixel array, a visible-light receiving element that receives visible light and an infrared-light receiving element that receives infrared light in at least a short-wavelength infrared band; and
a processing circuit that generates a first image illustrating an image of a dried food in a visible region and a second image illustrating an image of the dried food in an infrared region from data acquired by the imaging element, and inspects the dried food from the first image and the second image that have been generated.

2. The solid-state imaging device according to claim 1, wherein
the processing circuit detects a moisture content of the dried food from at least the second image.

3. The solid-state imaging device according to claim 2, wherein
the processing circuit compares the moisture content with a predetermined threshold and issues a notification that the dried food having the moisture content higher than the predetermined threshold is subject to a re-drying process.

4. The solid-state imaging device according to claim 3, wherein
the processing circuit calculates a drying time in the re-drying process on a basis of the moisture content.

5. The solid-state imaging device according to claim 2, wherein
the processing circuit infers the moisture content of the dried food by using a learned model.

6. The solid-state imaging device according to claim 1, wherein
the processing circuit detects a protein content of the dried food from at least the second image.

7. The solid-state imaging device according to claim 6, wherein
the processing circuit infers the protein content of the dried food by using a learned model.

8. The solid-state imaging device according to claim 1, wherein
the processing circuit detects a defect in appearance of the dried food from at least the first image.

9. The solid-state imaging device according to claim 8, wherein
the processing circuit detects foreign matter attached to the dried food from the first image.

10. The solid-state imaging device according to claim 8, wherein
the processing circuit detects deterioration of a color tone of the dried food from the first image.

11. The solid-state imaging device according to claim 8, wherein
the processing circuit performs an appearance inspection of the dried food by using a learned model.

12. The solid-state imaging device according to claim 1, wherein
the processing circuit inspects the dried food on a basis of the first image and the second image by using a learned model.

13. A solid-state imaging system comprising:
an infrared light source that emits infrared light including at least light in a short-wavelength infrared band; and
the solid-state imaging device according to claim 1,
wherein the imaging element causes the infrared-light receiving element to receive light emitted from the infrared light source and reflected by or transmitted through the dried food.
